SEQUENCE LISTING (11 sequences)

SEQ ID NO: 1 - protein, 8 aa, synthetic construct
  source 1..8: note = CDR1 in the heavy chain variable region
GYTFTDYA

SEQ ID NO: 2 - protein, 8 aa, synthetic construct
  source 1..8: note = CDR2 in the heavy chain variable region
FSTYYGNA

SEQ ID NO: 3 - protein, 10 aa, synthetic construct
  source 1..10: note = CDR1 in the light chain variable region
KSVSTSGYSY

SEQ ID NO: 4 - protein, 8 aa, synthetic construct
  source 1..8: note = CDR3 in the light chain variable region
QHIRELTR

SEQ ID NO: 5 - protein, 120 aa, synthetic construct
  source 1..120: note = heavy chain variable region of the mAb
QVQLKQSGAELVRPGVSVKISCKGSGYTFTDYAVHWVKQSHAKSLEWIGVFSTYYGNANYNQNFKGKATMTVDKSSNTAYMELARLTSEDSAIYYCARGGDYYGSSNVDYAMDYWGQGTS

SEQ ID NO: 6 - protein, 108 aa, synthetic construct
  source 1..108: note = light chain variable region of the mAb
ENVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWS

SEQ ID NO: 7 - DNA, 360 bp, synthetic construct
  source 1..360: mol_type = other DNA; note = DNA sequence encoding the heavy chain variable region of mAb
caggtgcaactgaagcagtctggggctgagctggtgaggcctggggtctcagtgaagatttcctgcaagggttctggctacacattcactgattatgctgtgcactgggtgaagcagagtcatgcaaagagtctagagtggattggagtttttagtacttactatggtaatgctaactacaaccagaacttcaagggcaaggccacaatgaccgtagacaaatcctccaacacagcctatatggaacttgccagactgacatctgaggattctgccatctattactgtgcaagaggaggggattactacggtagtagcaacgtagactatgctatggactactggggtcaaggaacctca

SEQ ID NO: 8 - DNA, 327 bp, synthetic construct
  source 1..327: mol_type = other DNA; note = DNA sequence encoding the light chain variable region of mAb
gaaaatgtgctcacccagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggagctga

SEQ ID NO: 9 - protein, 375 aa, synthetic construct
  source 1..375: note = SADS-CoV N protein
MATVNWGDAVEQAESRGRKRIPLSLFAPLRVIDGKNFWNVMPRNGVPTGKGTPDQQIGYWVEQKRWRMQKGQRKDQPSNWHFYYLGTGPHADAPFRKRIQGVHWVAVDGAKTSPTGLGVRNRNKEPATPQFGFQLPPDLTVVEVTSRSASRSQSRSRNQSQSRSGAQTPRAQQPSQSVDIVAAVKQALADLGIASSQSRPQSGKNTPKPRSRAVSPAPAPKPARKQMDKPEWKRVPNSEEDVRKCFGPRSVSRNFGDSDLVQHGVEAKHFPTIAELLPTQAALAFGSEITTKESGEFVEVTYHYVMKVPKTDKNLPRFLEQVSAYSKPSQIRRSQSQQDLNADAPVFTPAPPATPVSQNPAFLEEEVEMVDEIIN

SEQ ID NO: 10 - DNA, 1128 bp, synthetic construct
  source 1..1128: mol_type = other DNA; note = DNA sequence encoding the SADS-CoV N protein
atggccactgttaattggggtgacgctgttgaacaggcggaatctcgtggtcgtaaaagaattccattgtcactctttgcgcctttgcgtgttatagatggcaaaaacttttggaatgtcatgcctagaaatggagttccgacaggtaaaggcactccagatcaacagattggttattgggttgaacaaaaacgctggcgaatgcaaaaaggccaacgtaaagatcagccttctaactggcacttttattaccttggtactggtcctcacgcagatgctcctttcaggaaacggattcagggtgtgcattgggtcgctgttgacggtgctaaaactagccccacaggtcttggtgttcgcaatcgtaacaaagaacctgctacacctcagtttgggtttcaattaccaccagacctgactgttgttgaggttacttctagaagtgcttcacgttcacagtctcgttctcgcaatcaaagtcaaagccgcagtggtgctcagacacctcgtgctcaacagccgtcacagtctgttgacattgttgctgcagttaaacaagctttggcagacttgggcatagcttctagccagtccaggcctcaaagtggtaaaaatacacccaaaccaagaagcagagctgtctcacctgcacctgcccctaaaccggctcgtaagcagatggacaaacctgaatggaagcgtgttcctaattctgaggaggacgtgcgtaaatgctttggtcctcgctcagtttctagaaattttggtgacagtgacctcgttcagcacggtgttgaagctaagcactttccaacaattgctgagttgcttccgacacaagctgcactagcctttggtagtgaaatcacaaccaaagagtctggtgaatttgtagaagtcacctatcactatgtaatgaaggtccccaagactgataaaaatctacccagatttcttgagcaagtctcggcttactctaaacccagtcaaattaggagatctcaatctcaacaagacctaaatgctgatgccccagtgttcactccggcacctccagctactccagtttcccaaaatcctgcttttcttgaggaggaggttgagatggtggatgagattattaattag

SEQ ID NO: 11 - protein, 18 aa, synthetic construct
  source 1..18: note = CDR3 in the heavy chain variable region
ARGGDYYGSSNVDYAMDY

(19) United States
(12) Patent Application Publication
(10) Pub. No.: US 2023/0194526 A1
(43) Pub. Date: Jun. 22, 2023

(54) BLOCKING ELISA KIT FOR DETECTING ANTIBODY TO SWINE ACUTE DIARRHEA SYNDROME CORONAVIRUS N PROTEIN

(71) Applicant: Institute of Urban Agriculture, Chinese Academy of Agricultural Sciences, Chengdu City (CN)

(72) Inventors: QI WANG, Chengdu City (CN); LIYAN CAO, Chengdu City (CN); HAIXUE ZHENG, Chengdu City (CN); HONG TIAN, Chengdu City (CN); XIANGYU KONG, Chengdu City (CN); CONG YUAN, Chengdu City (CN); YUEYUE DUAN, Chengdu City (CN)

(21) Appl. No.: 17/860,961

(22) Filed: Jul. 8, 2022

(30) Foreign Application Priority Data
Nov. 30, 2021 (CN) 202111449947.7

(51) Int. Cl. G01N 33/569; C07K 14/005; C07K 16/10; C12N 15/70; G01N 33/535; G01N 33/543; G01N 33/577

(52) U.S. Cl. CPC G01N 33/56983; C07K 14/005; C07K 16/10; C12N 15/70; G01N 33/535; G01N 33/54306; G01N 33/577; C07K 2317/56; C07K 2317/565; C07K 2317/76; C12N 2800/101; G01N 2333/165; G01N 2469/20

(57) ABSTRACT

The disclosure belongs to the field of biotechnology, and in particular to a blocking ELISA kit for detecting an antibody to swine acute diarrhea syndrome coronavirus (SADS-CoV) N protein. The kit includes an enzyme plate coated with SADS-CoV N protein, an HRP-labeled mouse anti-SADS-CoV N protein monoclonal antibody (mAb), a positive serum control, and a negative serum control. The kit may detect positive sera diluted at 1:512, with no cross-reaction with positive sera against porcine epidemic diarrhea virus (PEDV), transmissible gasteroenteritis virus (TGEV) and porcine deltacoronavinis (PDCoV) etc., and the intrabatch and interbatch coefficient of variation is less than 10%. The comparison result with the indirect immunofluorescence test shows that the concordance rate of the blocking ELISA kit of the present disclosure is 99.6%, the Kappa value is 0.91, and the blocking ELISA method established by the present disclosure is highly consistent with IFA.

### CROSS REFERENCE TO RELATED APPLICATION

This patent application claims the benefit and priority of Chinese Patent Application No. 202111449947.7, entitled “BLOCKING ELISA KIT FOR DETECTING ANTIBODY TO SWINE ACUTE DIARRHEA SYNDROME CORONAVIRUS N PROTEIN” filed on Nov. 30, 2021, the disclosure of which is incorporated by reference herein in its entirety as part of the present application.

### TECHNICAL FIELD

A computer readable XML file entitled “GWP20220400016_seqlist.xml”, that was created on Nov. 14, 2022, with a file size of about 15,086 bytes, contains the sequence listing for this application, has been filed with this application, and is hereby incorporated by reference in its entirety.

### TECHNICAL FIELD

The disclosure belongs to the field of biotechnology, and in particular to a blocking ELISA kit for detecting an antibody to swine acute diarrhea syndrome coronavirus N protein.

### BACKGROUND ART

Swine acute diarrhea syndrome (SADS) is an acute infectious disease caused by swine acute diarrhea syndrome coronavirus (SADS-CoV), causing acute diarrhea and vomiting in infected piglets clinically, as well as rapid death of newborn piglets younger than 5 days old due to sudden weight loss and severe dehydration, with a nearly 90% fatal mortality SADS-CoV was isolated from fecal samples of piglets with severe diarrhea in a pig farm in Guangdong Province in 2017 by Chinese researchers. SADS-CoV is a newly discovered porcine enteric coronavirus. It has more than 90% nucleotide sequence homology to the bat-derived HKU2, and close evolutionary distance from human coronavirus 229E/NL63, both of which take human angiotensin-converting enzyme 2 (ACE2) as an invasion receptor, indicating that SADS-CoV has a potential threat of cross-species transmission to humans. Given that there are currently no effective vaccines and antiviral drugs available, the rapid and accurate diagnosis of SADS is of great significance for the prevention and control of the disease.

As an enveloped, single-stranded positive-sense RNA virus, SADS-CoV belongs to the genus Alphacoronavirus of the family Coronaviridae. The genome of SADS-CoV is approximately 27 kb in size, with a cap structure at the 5′ end and a Poly(A) tail at the 3′ end. Spike protein (S), envelope protein (E), membrane glycoprotein (M), and nucleocapsid protein (N) are the four structural proteins of SADS-CoV. The coding region of N gene is 1128 bases in length, encoding 375 amino acids in total, and the molecular mass of the protein is about 41.7 ku. The N protein is well conserved, has strong immunogenicity, and is expressed in the entire process of virus infection. It induces the body to produce neutralizing antibodies, and is a key protein for virus serological diagnosis and immunological detection.

Enzyme-linked immunosorbent assay (ELISA) has been widely used in the detection of human and animal diseases due to simple operation, strong specificity and high sensitivity. There are currently no commercial kits for SADS-CoV detection in the market. Peng et al. established an indirect ELISA method using purified SADS-CoV S protein as the coating antigen (Peng, P., Gao, Y., Zhou, Q., Jiang, T., Zheng, S., Huang, M., Xue, C., Cao, Y., Xu, Z., 2021. Development of an indirect ELISA for detecting swine acute diarrhoea syndrome coronavirus IgG antibodies based on a recombinant spike protein. Transboundary and emerging diseases). Compared with indirect immunofluorescence assay (IFA), the concordance rate of positive clinical samples was 97.8%, and that of negative clinical samples was 94.7%. This method has relatively high requirements for the coated antigen, otherwise false positives and negatives are prone to occur.

### SUMMARY

The present disclosure establishes a blocking ELISA kit for detecting a SADS-CoV serum antibody based on SADS-CoV N protein specific monoclonal antibody (mAb). Compared with indirect ELISA, the method has lower requirements for the purity of the coated antigen, and has the advantages of greater specificity, and higher sensitivity.

The blocking ELISA kit for detecting an antibody to SADS-CoV N protein of the present disclosure includes an ELISA plate coated with SADS-CoV N protein, and a horseradish peroxidase (HRP)-labeled mouse anti-SADS-CoV N protein mAb.

In some embodiments, a method for preparing the SADS-CoV N protein includes designing specific primers according to SADS-CoV GDSO4 strain N protein (Genbank accession number: MF167434.1), obtaining N gene through PCR amplification, conducting ligation to a prokaryotic expression vector pET32a to construct a recombinant plasmid pET32a-N, conducting transformation into Escherichia coli BL21 (DE3), inducing expression with IPTG to obtain an N protein, and then conducting purification using a nickel column to obtain a purified recombinant N protein. The amino acid sequence of the SADS-CoV N protein is set forth in SEQ ID NO: 10, and the DNA sequence encoding the SADS-CoV N protein is set forth in SEQ ID NO: 11.

In some embodiments, a method for preparing the mouse anti-SADS-CoV N protein mAb includes the following steps: immunizing a Balb/c mouse with the purified recombinant N protein as an immune source at a dose of 30 μg/mouse. For a first immunization, the purified recombinant N protein and Freund's complete adjuvant are mixed in equal volumes for emulsification, and subcutaneously injected at multiple points on the back. Boosts are performed every 2 weeks for a total of four immunizations. The boost includes mixing the purified recombinant N protein and Freund's incomplete adjuvant in equal volumes for emulsification, and conducting immunization in a way the same as the first immunization. After a fourth immunization, mouse spleen cells are fused with myeloma cells SP2/0 cells to prepare hybridoma cells, and cell supernatant is verified by indirect ELISA and IFA for positive clone screen. After 3 times of subcloning, the hybridoma cells are injected into mice to prepare ascites, and then an anti-mouse SADS-CoV N protein mAb is obtained.

The amino acid sequece of a heavy chain variable region of the mouse anti-SADS-CoV N protein mAb is set forth in SEQ ID NO: 6, in which the heavy chain variable region includes a CDR1 having the amino acid sequence set forth in SEQ ID NO: 1, a CDR2 having the amino acid sequence set forth in SEQ ID NO: 2, and a CDR3 having the amino acid sequence set forth in SEQ ID NO: 3. The amino acid sequence of a light chain variable region of the mouse anti-SADS-CoV N protein mAb is set forth in SEQ ID NO: 7, in which the light chain variable region includes a CDR1 having the amino acid sequence set forth in SEQ ID NO: 4, a CDR2 having the amino acid sequence Leu-Val-Ser and a CDR3 having the amino acid sequence set forth in SEQ ID NO: 5.

The DNA sequence encoding the heavy chain variable region of the mouse anti-SADS-CoV N protein mAb is set forth in SEQ ID NO: 8; the DNA sequence encoding the light chain variable region of the mouse anti-SADS-CoV N protein mAb is set forth in SEQ ID NO:10.

The blocking ELISA kit for detecting an antibody to SADS-CoV N protein of the present disclosure further includes a positive senim control and a negative serum control.

The positive serum control is swine serum collected after artificial immunization with SADS-CoV; the negative senim control is swine serum without SADS-CoV pathogen.

The blocking ELISA kit for detecting an antibody to SADS-CoV N protein of the present disclosure further includes a coating solution, a blocking solution, a sample diluent, an enzyme-labeled antibody diluent, a washing solution, a color development solution and a stop solution.

The coating solution is 0.05 M carbonate buffer (1.5 g Na₂CO₃, 2.92 g NaHCO₃, in deionized water to make up 1 L, pH 9.6), and the sample diluent is 1% BSA [0.9% NaCl, 0.5% Tween-20, 0.05 mol/L MOPS, pH 7.0], the enzyme-labeled antibody dilution may be purchased from Jrnan Baiditai Biotechnology Co., Ltd.: the washing solution is 20X PBST (5.4 g KH₂PO₄, 28.4 g Na₂HPO₄·12H₂O, 160 g NaCl, 4 g KCl, and 10 mL Tween-20 in deionized water to make up 1 L, pH 7.4); the color development solution is TMB, and the stop solution is 2M H₂SO₄.

The blocking solution is a PBST solution containing 5% skim milk, 5% BSA or 2% trehalose. 5% skim milk, 5% BSA and 2% trehalose, i.e., 5 g skim milk, 5 g BSA and 2 g trehalose per 100 mL of PBST solution, respectively.

A method for using the blocking ELISA kit for detecting an antibody to SADS-CoV N protein includes the following steps:

(1) coating: diluting a purified SADS-CoV N protein with a coating solution, subjecting an ELISA plate to coating overnight at 4° C., discarding liquid in the ELISA plate, conducting washing 4 times, and patting the ELISA plate dry with absorbent paper;

(2) blocking: adding a blocking solution, sealing the ELISA plate, discarding liquid in the plate, conducting washing 4 times, and patting the ELISA plate dry with absorbent paper;

(3) sample addition: adding a serum to be tested diluted with a sample diluent for reaction, providing negative control, positive control and blank control wells, discarding liquid in the plate, conducting washing 4 times, and patting the ELISA plate dry with absorbent paper;

(4) enzyme-labeled secondary antibody addition: adding an HRP-labeled anti-mouse SADS-CoV N protein mAb diluted with enzyme-labeled antibody diluent for reaction, discarding liquid in the ELISA plate, conducting washing 4 times, and patting the ELISA plate dry with absorbent paper;

(5) color development: adding TMB substrate to develop color in the dark;

(6) termination: adding 2M H₂SO₄ to terminate the reaction;

(7) reading: measuring OD₄₅₀ by microplate reader; and

(8) calculation of the plaque inhibition (PI): PI=(OD value of negative control−OD value of serum to be tested)/OD value of negative control×100%.

A test sample with PI≥47.49613% is positive, a test sample with PI<37.26795% is negative, and a test sample with 37.26795%<PI<47.49613% is suspected, indicating a repeated test is needed. If the PI is still lower than 47.49613%, the test sample is negtive.

In some embodiments, the SADS-CoV N protein is coated at a concentration of 0.25 μg/mL.

In some embodiments, the serum to be tested is diluted at ratio of 1:4.

In some embodiments, the HRP-labeled anti-mouse SADS-CoV N protein mAb is diluted at a ratio of 1:16000.

The beneficial effects of the embodiments of the present disclosure include:

In the present disclosure, a purified SADS-CoV N protein is used as antigen, a mouse anti-SADS-CoV N protein mAb is used as detection antibody, and a series of reaction conditions and reagent optimization are adopted to establish a blocking ELISA kit for dectecting a SADS-CoV N protein antibody and a method of detection related thereto. The blocking ELISA kit established by the present disclosure has good specificity, sensitivity, and intrabatch and interbatch repeatability. The kit may detect positive sera diluted at a ratio as high as 1:512, and does not cross-reacted with positive sera of porcine epidemic diarrhea virus (PEDV), transmissible gasteroenteritis virus (TGEV) and porcine deltacoronavirus (PDCoV), etc., with the intrabatch and interbatch coefficient of variation less than 10%.

The comparison result with the IFA test shows that the concordance rate of the blocking ELISA kit in the present disclosure is 99.6%, and the K value is 0.91, illustrating that the blocking ELISA method established in the present disclosure is highly consistent with IFA.

The blocking ELISA kit established in the present disclosure may be used for clinical detection of SADS-CoV N protein antibody, and provides a rapid method of detection and monitoring means for the prevention and control of SADS-CoV infection.

### BRIEF DESCRIPTION OF THE DRAWINGS

FIGS. 1A-B show a result of PCR amplification of SADS-CoV N gene and identification results of pET32a-N recombinant plasmid digestion;

FIG. 1A. M: DL2000 relative molecular mass standard; 1: N gene, about 1000 bp in size; 2: water control.

FIG. 1B. M: DL5000 relative molecular mass standard; 1: The pET32a-N recombinant plasmid double digested with BamH I and Xho I.

FIGS. 2A-B show the expression and purification results of pET32a-N recombinant protein;

FIG. 2A. M: Protein Marker; 1: Before pET32a-N induction; 2: pET32a-N lysis supernatant; 3: pET32a-N precipitation after lysis;

FIG. 2B. M: Protein Marker; 1-2: Purified pET32a-N protein.

FIG. 3 is a graph showing the titer of the anti-mouse SADS-CoV N protein polyclonal antibody detected by ELISA.

FIGS. 4A-B show an indirect immunofluorescence assay to verify the reactivity of the mAb.

FIG. 4A. 6E8 mAb, 500-fold dilution; FIG. 4B. SP2/0 cell supernatant.

FIG. 5 shows the reactivity of mAb identified by Western blot;

M: Protein Marker, 1: purified pET32a-N protein; 2: Huh7 cell control; 3: SADS-CoV infected Huh7 cells.

FIG. 6 shows the subclass identification of the mAb.

FIG. 7 shows the identification result of the mAb by SDS-PAGE after purification.

M: Protein Marker; 1: 6E8 mAb purification.

FIGS. 8A-G are identification results showing that the mAb can recognize the N protein region;

FIG. 8A. a schematic diagram of expression of truncated N protein;

FIG. 8B. SDS-PAGE identification of induced expression of R1 and R2 regions. M: protein marker; 1: before R1 induction; 2: R1 lysis supernatant; 3: R1 lysis precipitate; 4: before R2 induction; 5: after R2 induction;

FIG. 8C. Western blot identification of E8. M: protein marker; 1: R1 expressing bacteria; 2: R2 expressing bacteria;

FIG. 8D. SDS-PAGE identification of induced expression of R1.1 and R1.2 regions. M: protein marker; 1: before R1.1 induction; 2: after R1.1 induction; 3: before R1.2 induction; 4: after R1.2 induction;

FIG. 8E. Western blot identification of E8. M: protein marker; 1: R1.1 expressing bacteria; 2: R1.2 expressing bacteria;

FIG. 8F. SDS-PAGE identification of induced expression of R1.2.1 and R1.2.2 regions. M: protein marker; 1: before R1.2.1 induction; 2: after R1.2.1 induction; 3: before R1.2.2 induction; 4: after R1.2.2 induction;

FIG. 8G. Western blot identification results of E8. M: protein marker; 1: bacteria expressing R1.2.1; 2: bacteria expressing R1.2.2;

FIGS. 9A-B are the PCR amplification of the variable region of SADS-CoV N protein mAb;

FIG. 9A. PCR amplification of the variable region of the heavy chain. M: DL2000 marker; 1: water control; 2: PCR amplification of 6E8 heavy chain variable region.

FIG. 9B. PCR amplification of the light chain K variable region. M: DL2000 marker; 1: water control; 2: 6E8 light chain variable region.

### DETAILED DESCRIPTION OF THE EMBODIMENTS

The present disclosure will be described in detail below through specific embodiments, so as to facilitate the understanding of the technical solutions of the present disclosure, but it is not intended to limit the protection scope of the present disclosure.

SADS-CoV strain GDS04 was gifted by Professor Yongchang Cao of Sun Yat-sen University. The giver isolated the strain according to Gong L., et al. A new bat-HKU2-like coronavirus in swine, China, 2017. Emerging Infectious Diseases, 2017, 23(9).
1. Construction, Expression and Purification of SADS-CoV N Protein Recombinant Plasmid
1.1 Construction of pET32a-N Recombinant Plasmid

Specific primers were designed using the SADS-CoV GDSO4 strain N protein (Genbank accession number: MF167434.1) as a reference. The upstream primer was P1: 5′-CGCGGATCCATGGCCACTGTTAATTGG-3′, and the downstream primer was P2: 5′-CCGCTCGAGCTAATTAATAATCTCATC-3′, in which BamHI and Xhol restriction sites (underlined) were introduced at the 5′ end of the upstream and downstream primers. After primer synthesis, PCR amplification was performed using the cDNA of SADS-CoV GDS04 strain as a template. The PCR reaction system included 25 μL of PrimeSTAR Max Premix (2×), 1 μL of P1 and P2 each, 1 μL of cDNA, and a balance of ddH₂O to 50 μL. The reaction program was as follows: pre-denaturation at 98° C. for 2 min; denaturation at 98° C. for 10 s, annealing at 55° C. for 30 s, extension at 72° C. for 2 min, 30 cycles; and extension at 72° C. for 10 min. PCR products were detected by 1% agarose gel electrophoresis. Gel extraction was perfonned according to the instruction of the Omega's EZNAGel Extraction Kit to obtain a gel-extracted N gene PCR product.

The gel-extracted N gene PCR product and pET32a vector were digested with BamHI and XhoI, then ligated and transformed into DH5α competent cells. The constructed recombinant plasmid was identified by double-enzyme digestion, and the plasmid identified as positive by enzyme digestion was verified by sequencing, and the positive recombinant plasmid was named pET32a-N.

1.2 Induced Expression and Purification of Recombinant N Protein

The recombinant plasmid pET32a-N was transformed into E. coli BL21 (DE3), and a single colony was picked and placed in 5 mL of ampicillin-resistant LB. When the OD₆₀₀ was about 0.8, IPTG with a final concentration of 1 mmol/L was added to induce expression. 5 h after induction, the precipitate was collected by centrifugation at 12,000 rpm for 2 min. The precipitate was resuspended with PBS, and then sonicated for 5 min (ultrasonic for 3 s, paused for 3 s). After sonication, the cells were centrifuged at 12,000 rpm for 10 min at 4° C., and supernatant and pellet were collected. 5× loading buffer were added to the supernatant and pellet, and a resulting mixture was boiled in boiling water for 10 mM, and identified by SDS-PAGE.

The induced expression of pET32a-N protein was expanded according to the above method, and the supernatant after ultrasonication lysis was purified by nickel column. The steps were as follows:

1) resin loading: an empty column was taken to add 2 mL of nickel column NTA resin; when the preservation solution dropped to the surface of the resin, the column was washed once with 5 times the column volume of distilled water, and then the column was balanced with 5 times the column volume of balance solution (20 mM Tris-HCl, 500 mM NaCl, 5 mM imidazole, pH 7.4);

2) loading: when the balance solution dropped to the surface of the resin, 3 mL of the lysis supernatant containing recombinant protein was added; the loading was repeated 2 to 3 times for 2 mM each time, and fluid flow-through was collected; the cokm-in was rinsed once with 5 times the column volume of balance solution;

3) protein elution: the protein sample was eluted with 75 mM imidazole in the balance solution.

4) cokmrn cleaning and storage: the column was rinsed once with 5 times the column volume of 0.5 M NaOH, and once with distilled water, then 70% absolute ethanol was added, and stored in a 4° C. refrigerator; each collected sample was identified by SDS-PAGE.
2. Preparation of Mouse Anti-SADS-CoV N Protein mAb
2.1 Mouse Immunization

Babl/c mice were immunized with purified recombinant N protein at 30 μg/mouse. For the first immunization, the N protein and Freund's complete adjuvant were mixed in equal volume and then emulsified, and subcutaneously injected at multiple points on the back. Boost was performed every 2 weeks (2W) for a total of four immunizations. The boost included mixing equal volumes of recombinant N protein and incomplete Freund's adjuvant for emulsification. The immunization method was the same as that of the first immunization.
2.2 ELISA for Detection of Antibody Titer

1 week after the fourth immunization, the mice were tailed and blood was collected to determine the antibody titer. The ELISA plate was coated with the inactivated SADS-CoV virus solution and coating solution diluted at 1:1 overnight at 4° C. at 50 gL/well, and washed four times with PBST. 2% trehalose was added for blocking at 4° C. for 10 h. The positive serum of mice immunized with N protein and the negative serum of mice not immunized with N protein were serially diluted with PBST from 1:100 to 1:12,800, with 8 gradient dilutions. HRP-labeled goat anti-mouse IgG (1:20,000 dilution) was added for reaction at 37° C. for 30 mM, washed four times with PBST, then TMB was added for color development, and OD₄₅₀ was measure on a microplate reader.
2.3 Preparation of the mAb

The steps were as follows:

(1) preparation of feeder layer cells: the HAT medium was injected into the abdominal cavity of mice, and aspirated slowly and repeatedly, and then a resulting liquid was spread in a 96-well plate;

(2) cell fusion: spleen cells and SP20 cells were fused under the action of the fusion agent PEG; the fused cells were plated in 96-well plates containing feeder cells;

(3) screening of positive clones:

a) indirect ELISA detection method: the purified N protein (2 μg/mL) was diluted with coating solution and then coated on ELISA plate at 50 μL/well overnight at 4° C., washed four times with PBST, and 2% Trehalose was added for blocking at 4° C. for 10 h; the supernatant of the fused cells was added to the ELISA plate, and incubated at 37° C. for 30 min; the plate was washed four times with PBST, then HRP-labeled goat anti-mouse IgG (1:20,000 dilution) was added, and the cells were incubated at 37° C. for 30 min; TMB was added for color development, and OD₄₅₀ was measured on a microplate reader; and

b) IFA method: Huh7 cells were infected with SADS-CoV; 36 h after virus infection, cells were fixed with paraformaldehyde; after the cell fixation, supernatant from ELISA antibody-positive cell wells was added to virus-infected cells, and then incubated with FITC-anti-mouse secondary antibody (100-fold dilution); the cells were observed under a fluorescence microscope after the reaction.

(4) cloning of positive hybridoma cells: positive wells as identified by ELISA and IFA was selected for subcloning of the positive hybridoma cells using the limiting dilution method; after 7-10 days of cell culture, the cells were observed under an inverted microscope, and the supernatant in wells with only a single clone growing was taken for antibody detection using the above ELISA and IFA methods; positive cells were taken for the next round of subcloning for a total of three times; and

(5) preparation of ascites: Balb/c mice of 10-12W were taken, with each mouse intraperitoneally injected with 0.5 mL of incomplete Freund's adjuvant and 5×10⁵ hybridoma cells (0.2 mL) were intraperitoneally injected into each mouse after 1 W; after 7 to 10 days, the abdomen of the mouse was obviously raised, then the ascites was collected, the titer was detected by ELISA, and the aliquots were stored at −80° C.
3. Identification of mAb
3.1 Specificity Identification

ELISA, IFA and western blot validation were included.

(1) ELISA verification: the purified N protein and His-tagged irrelevant protein (2 μg/mL) were diluted with coating solution and then coated on ELISA plate at 50 μL/well overnight at 4° C. The plate was washed four times with PBST, and after that, 2% trehalose was added for blocking at 4° C. for 10 h. The mAb was added to the plate coated with the N protein and His-tagged irrelevant protein, and incubated at 37° C. for 30 min. After the plate was washed four times with PBST, HRP-labeled goat anti-mouse IgG (1:20,000 dilution) was added, and incubated at 37° C. for 30 min. After the plate was washed four times with PBST, TMB was added for color development, and OD₄₅₀ was measured on a microplate reader.

(2) IFA verification: The ascites was diluted with PBS (500 times) and added to virus-infected cells, followed by incubation with FITC-anti-mouse secondary antibody (100 times dilution), and observed under a fluorescence microscope after the reaction.

(3) Western blot verification: the purified N protein or virus-infected and uninfected Huh7 cells were taken for SDS-PAGE, and then a resulting protein gel was transferred to NC membrane for Western blot verification. The primary antibody is mAb (1: 5000 dilution), and the secondary antibody is HRP-anti-mouse IgG (1:20,000 dilution).
3.2 Subclass Identification

The monoclonal antibodies obtained were identified by the SBA ClonotypingTM System/HRP Antibody Subclass Identification Kit (Southern Biotech Co., Ltd).
3.3 Epitope Identification

The N gene was truncated and cloned into the prokaryotic expression vector pET32a. After expression induction, SDS-PAGE was performed and a resulting gel was transferred to an NC membrane. The reactivity of the mAb was verified by western blot to confirm the antigenic epitopes recognized by the mAb.
4. PCR Amplification and Sequence Determination of mAb Variable Region Gene

First, the RNA of mAb produced by hybridoma cells was extracted, and cDNA was synthesized by reverse transcription using Oligo-dt or random primers respectively (PrimeScript II 1st Strand cDNA Synthesis Kit, TAKARA, 6210A).

The antibody variable region gene was amplified by nested PCR. First, using the above cDNA as a template, the first round of murine antibody IgG1 and κ light chain primers were used to amplify the variable region gene of the antibody, and then the first round of product was used as a template, and the second round of murine antibody IgG1 and κ light chain primers were used for amplification of the antibody variable region gene. The PCR reaction system included 25 μL of PrimeSTAR Max Premix (2×), 1 μL of P1 and P2 each, 1 μL of cDNA, a balance of ddH₂O to 50 μL. The reaction program was as follows: pre-denaturation at 98° C. for 2 min; denaturation at 98° C. for 10 s, annealing at 55° C. for 30 s, extension at 72° C. for 30s, 30 cycles; extension at 72° C. for 10 min. Primers for amplification of the antibody variable region gene could be found in the Reference (von Boehmer, L., Liu, C., Ackerman, S., Gitlin, AD, Wang, Q., Gazumyan, A., Nussenzweig, M. C., 2016. Sequencing and cloning of antigen-specific antibodies from mouse memory B cells. Nature protocols 11, 1908-1923.)

After amplification, 1% agarose gel electrophoresis was carried out. The gene size of the variable region of heavy chain and κ light chain was about 300 bp, and the target fragment was retrieved by gel extraction. The gel-extracted target fragment was inserted into the pMD-19T vector for sequence determination.
5. Purification of mAb

Affinity chromatography purification was carried out according to the instruction of NAbTM Protein G Spin Purification Kit (PIERCE Co., Ltd), and then SDS-PAGE was performed to confirm the purity.
6. HRP-Labeled Anti-Mouse SADS-CoV N protein mAb

Anti-mouse SADS-CoV N protein mAb was labeled according to the instruction of Peroxidase Labeling Kit (Roche), and the labeling effect of the mAb was detected by ELISA.
7. Establishment of Blocking ELISA Method for Detecting SADS-CoV N protein Antibody
7.1 Serum Preparation

(1) Preparation of SADS-CoV positive swine serum (standard positive serum): healthy piglets of 35 days old were selected for the first immunization with 2 mL of SADS-CoV (10⁴ TCID₅₀/inl) by Houhai point injection; after an interval of 2 weeks, 2 times the first immune dose of inactivated virus were injected to Houhai point; 2 weeks later, a boost with the same immunization dose as the second immunization was performed. One week after the boosts, the sera were isolated and identified as SADS-CoV positive by blocking ELISA and IFA.

(2) Preparation of SADS-CoV negative pig serum (standard negative serum): the serum was prepared from blood collected from negative pigs without SADS-CoV infection as identified by blocking ELISA and IFA.
7.2 Selection of Optimal Reaction Conditions for Blocking ELISA
7.2.1 Determination of Optimal Antigen Coating Concentration and Optimal Dilution of Serum Samples

(1) SADS-CoV N protein was diluted at the ratio of 4, 2, 1, 0.5, 0.25, 0.125, 0.0625, and 0.03125 ug/mL with the coating solution, and added to the ELISA plate laterally at 50 μL each well for coating overnight at 4° C. After drying, the plate were washed 4 times with PBST on a plate washer.

(2) 2% trehalose was added for blocking at 37° C. for 1 h. After drying, the plate was washed three times by the same method as that in step (1).

(3) The negative and positive serum were serially diluted at 1:2, 1:4, 1:8, 1:16, 1:32, and 1:64, and added to the ELISA plate vertically for reaction at 37° C. for 30 min. After drying, the plate was washed three times by the same method as that in step (1).

(4) The supernatant of HRP-labeled 6E8 mAb (HRP-6E8) diluted with antibody diluent (1: 8,000) was added to the ELISA plate at 50 μL per well, and incubated at 37° C. for 30 min. After drying, the plate was washed three times by the same method as that in step (1).

(5) 50 μL of TMB substrate chromogenic solution was added to each well for reaction at 37° C. for 10 min.

(6) 50 μL of 2M H₂SO₄ was added to each well to stop the reaction. The OD₄₅₀ₙₘ value was read to calculate the PI value. When the PI value is the largest, the coating concentration and the dilution concentration of the serum were the optimal working concentrations.
7.2.2 Determination of Optimal Blocking Solution and Working Conditions

The ELISA plate was coated overnight at 4° C. according to the optimal working concentration of SADS -CoV N protein determined in 7.2.1. 5% skim milk, 5% BSA and 2% trehalose were selected as the blocking solution for reaction at 37° C. for 1 h. The best blocking solution was determined according to OD₄₅₀ₙₘ value and PI value.

The ELISA plate was blocked with the best blocking solution, and blocking was carried out at 37° C. for 1 h, 2 h, 3 h and 4 h, as well as overnight at 4° C. The optimal blocking conditions were determined according to the OD₄₅₀. value and the PI value.
7.2.3 Determination of Optimal action time of serum to be tested

After the ELISA plate was coated and blocked under the optimal conditions, the serum to be tested was added at the optimal dilution concentration for reaction at 37° C. for 15 min, 30 min, 45 min and 60 min. The optimal sample reaction time was determined according to the OD₄₅₀ₙₘ value and the PI value.
7.2.4 Working Concentration and Reaction Time of Enzyme-Labeled Antibody

After the serum to be tested was subjected to the optimal conditions for coating and blocking, the ELISA plate was washed, and the HRP-6E8 mAb was diluted with antibody dilution solution by 1:1,000, 1:2,000, 1:4,000, 1:8,000, 1:16,000, 1:32,000, 1:64,000, and 1:128,000 at 37° C. for 30 min. The optimal working concentration of the enzyme-labeled antibody was determined according to OD₄₅₀ₙₘ value and PI value.

After the optimal working concentration of enzyme-labeled antibody was determined, the serum was subjected for reaction at 37° C. for 15 min, 30 min, 45 min and 60 min. The reaction time of the enzyme-labeled antibody was determined according to the OD₄₅₀ₙₘ value and the PI value.
7.2.5 Determination of Color Development Time

After the enzyme-labeled antibody stood reaction under the optimal reaction conditions, the ELISA plate was washed, and the color was developed at 37° C. for 5 min, 10 min, 15 min and 20 min. The optimal substrate action time was determined according to the OD₄₅₀ₙₘ value and the PI value.
7.3 Determination of critical value

100 serum samples that were SADS-CoV negative as identified by IFA were detected according to the optimized blocking ELISA method, and their OD₄₅₀ₙₘ was measured after the detection, and the blocking rate (PI value), the average value of PI (x̅) and stand deviation (SD) of 100 negative serum samples were calculated. When the PI value of the sample is ≥x̅+3SD, the sample is judged as positive; when the PI value of the sample is ≤x̅+2SD, the sample is judged as negative; if the PI value is between the two, the sample is judged as suspicious and needs to be tested again, and if the sample is still suspicious, it is judged as negative.
7.4 Sensitivity Test

According to the screening reaction conditions of blocking ELISA, the sensitivity of the established blocking ELISA was tested by 2-fold dilution of SADS-CoV negative and positive standard serum samples.
7.5 Specificity Test

The optimized blocking ELISA method was used to detect PEDV, TGEV and PDCoV positive sera. Three replicates were set for each sample, and SADS-CoV negative and positive sera were used as controls.
7.6 Repeatability test

6 serum samples (3 positive sera and 3 negative sera each) were selected for testing to evaluate the intrabatch and interbatch repeatability of the blocking ELISA method, and the PI value was calculated based on the value of OD₄₅₀ₙₘ. Then, the coefficient of variation (CV=(SD/x̅)×100%) was calculated according to the PI value.
7.7 Comparative test of blocking ELISA detection method and IFA

The blocking ELISA method for SADS-CoV antibody detection established in this study and the IFA test were used to detect 246 field swine senim samples, and then the concordance rate between the blocking ELISA method and the IFA test results was calculated.
8. Results
8.1 Amplification of N Gene and Construction of Recombinant Plasmid

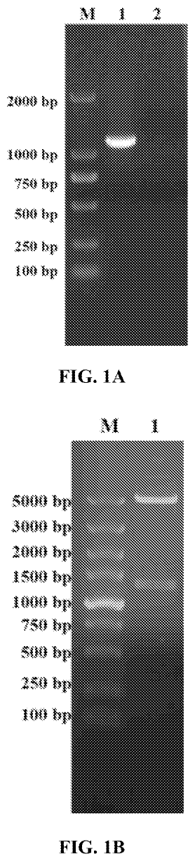

The N gene was amplified using SADS-CoV cDNA as a template, and the PCR product was verified by 1% agarose gel electrophoresis. As shown in FIG. 1A, the size of the N fragment was about 1,000 bp.

The gel-extracted N gene PCR product and pET32a vector were digested with BamHI and Xhol, and then ligated and transformed into DH5a competent cells. The constructed recombinant plasmid pET32a-N was identified by double-enzyme digestion. The digestion results are shown in FIG. 1B. The double-enzyme digestion products showed bands at more than 5,000 bp (vector band) and around 1,000 bp (target fragment) as expected. The plasmid identified as positive by enzyme digestion was confirmed by sequencing, and the sequencing results were consistent with the target sequence.

The sequence of the N gene is set forth in SEQ ID NO: 11.
8.2 Induced Expression and Purification of Recombinant N Protein

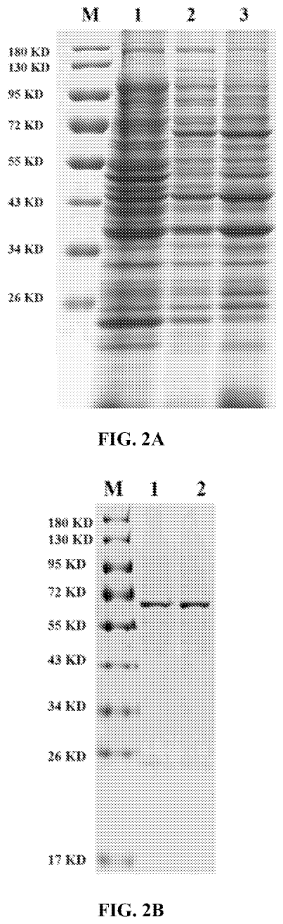

The recombinant plasmid pET32a-N was transformed into E. coil BL21 (DE3), and IPTG with a final concentration of 1 mmol/L was added to induce expression. The induced product was ultrasonicated and then identified by SDS-PAGE. It was shown that pET32a-N was expressed in both the supernatant and pellet (FIG. 2A), indicating that part of the N protein was soluble. Therefore, the protein was purified by nickel column purification. It could be seen from FIG. 2B that N protein with high purity was obtained. The amino acid sequence of the N protein is set forth in SEQ ID NO: 10.
8.3 Detection of Titer of SADS-CoV N protein Polyclonal Antibody by ELISA

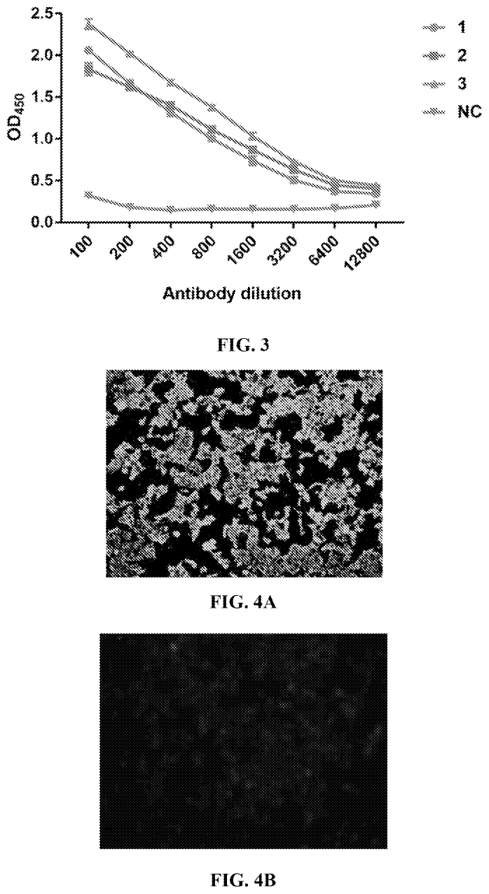

Balb/c mice were immunized with purified recombinant N protein, and the antibody titer was detected 1 week after the fourth immunization. The ELISA plate was coated with inactivated SADS-CoV virus solution, and the antibody titer was detected. The non-immunized mouse senim was used as the negative control (NC). When the sera were diluted at 1:12,800, the OD₄₅₀/NC of NOs. 1, 2, and 3 mouse sera was >2.0, indicating that the titer of the antibody could reach more than 1:12,800 (FIG. 3).
8.4 Detection of mAb Specificity

Through cell fusion technology, a mAb that specifically recognizes SADS-CoV N protein was screened and named 6E8. The IFA results showed that the 6E8 mAb was able to detect specific fluorescent signals in SADS-CoV-infected Huh? cells (FIG. 4A), while S/P20 cell supernatants SADS-CoV-infected cells did not show any fluorescence signals (FIG. 4B).

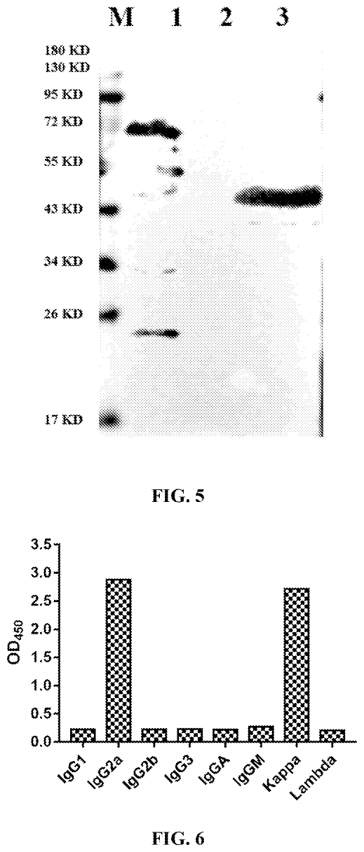

The ELISA results showed that the 6E8 mAb reacted with the N protein, but did not react with the His-tagged unrelated protein, indicating that the 6E8 mAb specifically recognized the N protein (Table 1). Western blot results showed that 6E8 could recognize the purified N protein as well as the N protein in virus-infected cells (FIG. 5).

TABLE 1
mAb ELISA test

| | OD₄₅₀ | |
|---|---|---|
| | | His-tagged |
| Sample | N protein | unrelavant protein |
| 6E8 | 2.89 | 0.218 |

8.5 Subclass Identification of 6E8 mAb

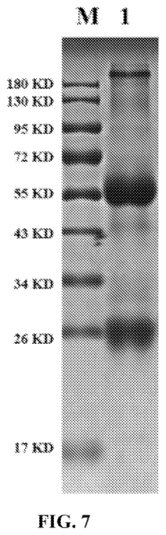

6E8 mAb was subtyped using the mouse mAb subclass identification kit. The identification results showed that the heavy chain constant region of the 6E8 mAb was IgG 2a subclass, and the light chain constant region was lc subclass (FIG. 6).
8.6 Antibody Purification

6E8 ascites was combined with Protein G for further separation and purification. The eluted product was identified by SDS-PAGE, and the result showed that a relatively pure 6E8 mAb was obtained, as shown in FIG. 7.
8.7 Identification of N Protein Region that 6E8 mAb Recognizes

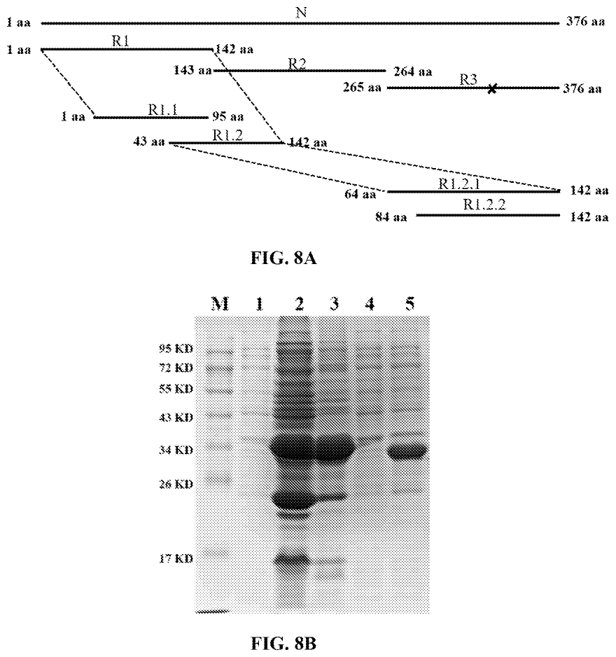

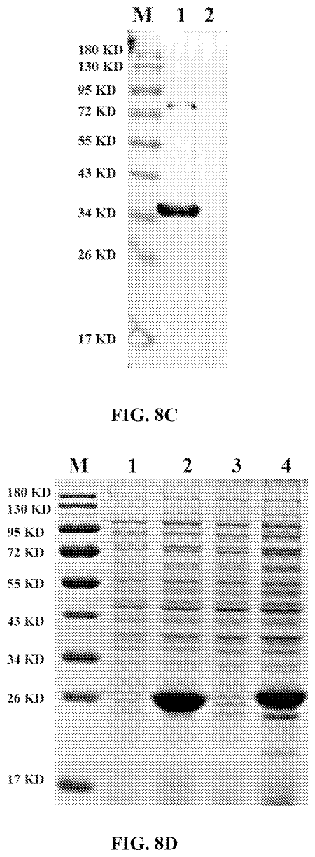

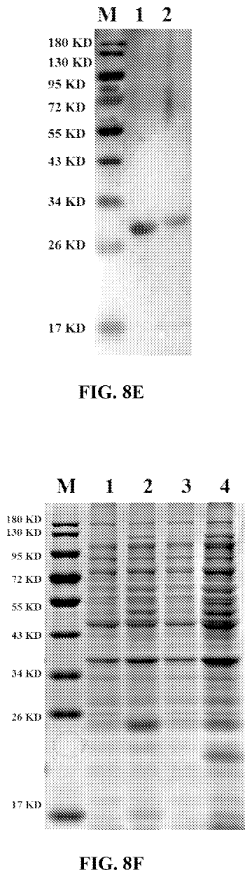

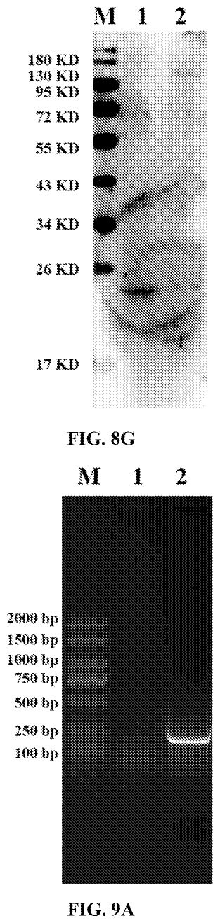

To identify the N protein region that the mAb recognizes, the truncated N proteins, named as RI, R2, R3, R1.1, R1.2, R1.2.1 and R1.2.2 regions respectively, was subjected to recombinant plasmids as shown in FIG. 8A. Insertion of the R3 region into the prokaryotic expression vector pET32a did not induce the expression of this protein, while all other regions were expressed (FIG. 8B). Western blot results showed that the 6E8 mAb recognized the R1 region but could not recognize the R2 region (FIG. 8C). Next, the R1 region was truncated. As shown in FIG. 8D and FIG. 8E, the 6E8 mAb could recognize the R1.1 and R1.2 regions, indicating that the 6E8 mAb recognized the N protein at the overlapping part of R1.1 and R1.2 regions, that is, 43 to 95 aa. The R1.2 region was further truncated. As shown in FIG. 8F and FIG. 8G, 6E8 only reacted with R1.2.1, but not with R1.2.2. Therefore, the recognition region of 6E8 mAb is a region that does not overlap with R1.2.2, that is, 64 to 84 aa.
8.8 PCR Amplification of Variable Region of 6E8 mAb

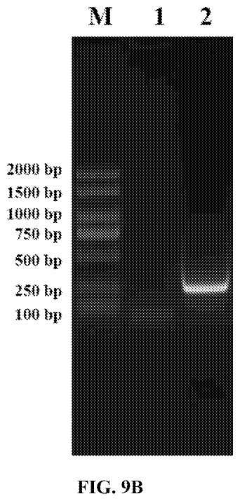

A PCR product with a size of about 300 bp was amplified from the cDNA of 6E8 hybridoma cells (FIGS. 9A-B), which was consistent with the expected size of the amplified product. After gel extraction, it was cloned into the pMD19T vector for sequencing. The sequencing results were aligned to the antibody gene library (IMGT), and the sequencing results confirmed that the amplified sequences were the DNA sequence of the heavy chain variable region and the DNA sequence of the light chain variable region of the mAb. Specifically, the DNA sequence encoding the heavy chain variable region of the anti-mouse SADS-CoV N protein mAb 6E8 is set forth in SEQ ID NO: 8; the DNA sequence encoding the light chain variable region of the anti-mouse SADS-CoV N protein mAb 6E8 is set forth in SEQ ID NO: 9.
8.9 Selection of Optimal Coating Concentration of Antigen and Optimal Dilution of Serum

According to the checkerboard method, when the PI value was the largest, the optimal coating concentration of SADS-CoV N protein was 0.25 ug/mL, and the optimal serum dilution was 1:4 (Table 2).

TABLE 2
Selection of optimal coating concentration
of antigen and optimal dilution of serum

| Antigen | | | | | | |
|---|---|---|---|---|---|---|
| coating | | | | | | |
| amount | Blocking rate at different serum dilutions (PI %) | | | | | |
| (μg/mL) | 1:2 | 1:4 | 1:8 | 1:16 | 1:32 | 1:64 |
| 4 | 81.04499 | 76.33721 | 43.83642 | 0.176991 | −0.61064 | 81.04499 |
| 2 | 85.4902 | 71.96726 | 28.53567 | 5.994199 | 1.017442 | 85.4902 |
| 1 | 89.68664 | 80.93797 | 57.13043 | 36.40747 | 20.52164 | 89.68664 |
| 0.5 | 90.805 | 88.2243 | 77.63955 | 63.92623 | 49.56986 | 90.805 |
| 0.25 | 89.74359 | 91.91307 | 87.03448 | 83.57283 | 77.14918 | 89.74359 |
| 0.125 | 81.25 | 86.77772 | 86.05457 | 89.18376 | 86.64137 | 81.25 |
| 0.0625 | 77.46741 | 87.96209 | 88.97005 | 89.14826 | 88.28039 | 77.46741 |
| 0.03125 | 63.97695 | 74.01247 | 76.18403 | 84.52752 | 84.03116 | 63.97695 |

8.10 Determination of Optimal Blocking Solution and Blocking Time

The blocking effect of 5% skim milk, 5% BSA and 2% trehalose in the same blocking time was compared. It was shown that when 2% trehalose was used for blocking, the PI value of the detected sample was the largest, reaching 92.1669%. Therefore, 2% trehalose had the best blocking effect (Table 3), and the best blocking time was overnight at 4° C. (Table 4). 5% skim milk, 5% BSA and 2% trehalose mean that 5g skim milk, 5g BSA and 2g trehalose are added to each 100mL PBST solution, respectively.

TABLE 3
Selection of the best blocking solution

| Blocking solution | 5% skim milk | 5% BSA | 2% trehalose |
|---|---|---|---|
| PI % | 91.90346 | 91.7503 | 92.1669 |

TABLE 4
Determination of optimal blocking time

| Blocking | 37° C. | | | | 4° C. |
|---|---|---|---|---|---|
| time | 1 h | 2 h | 3 h | 4 h | overnight |
| PI % | 86.27566 | 87.89286 | 87.64692 | 87.55237 | 90.54311 |

8.11 Determination of Optimal Action Time Of Sera to Be Tested

As shown in Table 5, when the action time of the sera to be tested was 45 min, the PI value was the largest. Therefore, the optimal action time of the serum to be tested was 45 min.

TABLE 5
Determination of the action time of the serum to be tested

| Serum action | | | | |
|---|---|---|---|---|
| time | 15 min | 30 min | 45 min | 60 min |
| PI % | 66.27204 | 91.06746 | 91.54039 | 88.81386 |

8.12 Selection of Working Concentration and Action Time of Enzyme-Labeled Antibody

As shown in Table 6, when HRP-6E8 was diluted at 1: 16,000, the PI value was the largest. Therefore, the optimal working concentration of enzyme-labeled antibody was 1: 16,000 times, and the optimal reaction time was 30 min (Table 7).

TABLE 6
Optimal working concentration of enzyme-labeled antibody

| | Dilution of HRP-6E8 | | | | | | | |
|---|---|---|---|---|---|---|---|---|
| | 1:1,000 | 1:2,000 | 1:4,000 | 1:8,000 | 1:16,000 | 1:32,000 | 1:64,000 | 1:128,000 |
| PI % | 49.81756 | 73.86187 | 78.35544 | 87.74523 | 89.81961 | 89.23611 | 85.94515 | 78.14748 |

TABLE 7
The optimal action time of enzyme-labeled antibody

| HRP-6E8 action | | | | |
|---|---|---|---|---|
| time | 15 min | 30 min | 45 min | 60 min |
| PI % | 84.99392 | 89.34316 | 87.98821 | 87.18225 |

8.13 Color Development Time

As shown in Table 8, when the color development time was 15 min, the PI value was the largest.

TABLE 8
Determination of color development time

| Color | | | | |
|---|---|---|---|---|
| development | | | | |
| time | 5 min | 10 min | 15 min | 20 min |
| PI % | 82.94612 | 85.92688 | 88.09321 | 86.93102 |

8.13 Determination of critical value

100 SADS-CoV antibody negative samples were detected, and PI values thereof were recorded. The mean value of PI was calculated to be 16.81161, and the standard deviation (SD) was 10.22817. According to the formula, x̅+3SD=47.49613, x̅+2SD=37.26795; therefore, Detected samples with a PI≥47.49613% were judged positive, samples with a PI≤37.26795% were judged negative, and samples with 37.26795%<PI<47.49613% were suspected.
8.14 Sensitivity Test

The sensitivity of the established blocking ELISA was tested by 2-fold dilution of SADS-CoV negative and positive sera. When the serum was diluted at 1:512, its PI value was 49.13924%, which was ueater than the critical value. Therefore, the sensitivity of this method reached 1: 512 (Table 9).

TABLE 9
Sensitivity of the blocking ELISA method

| Dilution factor | PI % |
|---|---|
| 2 | 69.25055 |
| 4 | 74.64051 |
| 8 | 81.87506 |
| 16 | 83.9083 |
| 32 | 82.33999 |
| 64 | 80.78093 |
| 128 | 74.98129 |
| 256 | 66.65515 |
| 512 | 49.13924 |
| 1024 | 35.3934 |
| 2048 | 23.54828 |
| 4096 | 16.11132 |

8.15 Specificity Test

As shown in Table 10, the optimized blocking ELISA method was used to detect SADS-CoV, PEDV, TGEV and PDCoV positive serum samples, and it was found that only SADS-CoV serum samples were positive, and there was no cross-reaction with the rest, indicating the method established by the present disclosure had good specificity.

TABLE 10
Specificity of the blocking ELISA method

| Serum sample | PI % |
|---|---|
| PEDV | 7.315931 |
| TGEV | 6.590191 |
| PDCoV | 10.52324 |
| NC | 6.941355 |
| SADS-CoV | 70.87674 |

8.16 Repeatability Test

The established blocking ELISA method was tested for intra- and inter-assay repeatability. The results are shown in Table 11. The coefficients of variation of the intrabatch and interbatch repeatability tests were both below 10%, indicating that the established blocking ELISA method had good repeatability.

TABLE 11
Reproducibility of the blocking ELISA method

| | 1 | | | 2 | | | 3 | | |
|---|---|---|---|---|---|---|---|---|---|
| | Batch | | | | | | | | |
| Serum | First | Second | Third | First | Second | Third | First | Second | Third |
| number | batch | batch | batch | batch | batch | batch | batch | batch | batch |
| 1 | 82.42806 | 82.77989 | 82.10143 | 50.40014 | 60.00949 | 49.58568 | 66.03099 | 67.74194 | 65.34637 |
| 2 | 85.33969 | 84.48767 | 85.0348 | 49.12311 | 62.28653 | 52.41962 | 69.811 | 69.59203 | 67.882 |
| 3 | 81.45752 | 82.82732 | 87.52072 | 55.15069 | 60.91082 | 53.46371 | 70.01532 | 69.82922 | 65.34637 |
| x̅ | 83.07509 | 83.36496 | 84.88565 | 51.55798 | 61.06894 | 51.823 | 68.6191 | 69.0544 | 66.19158 |
| SD | 1.649606 | 0.79411 | 2.214929 | 2.593375 | 0.936298 | 1.638441 | 1.831974 | 0.933088 | 1.195308 |
| Intra- | 1.985681 | 0.952571 | 2.609309 | 5.030016 | 1.533182 | 3.16161 | 2.669772 | 1.351236 | 1.805831 |
| batch | | | | | | | | | |
| CV % | | | | | | | | | |
| Inter- | 0.947834 | | | 8.067567 | | | 1.853496 | | |
| batch | | | | | | | | | |
| CV % | | | | | | | | | |
| | 4 | | | 5 | | | 6 | | |
| | Batch | | | | | | | | |
| Serum | First | Second | Third | First | Second | Third | First | Second | Third |
| number | batch | batch | batch | batch | batch | batch | batch | batch | batch |
| 1 | 5.704069 | 6.973435 | 6.231356 | 8.462455 | 8.064516 | 8.766987 | 9.177592 | 8.823529 | 8.617832 |
| 2 | 5.397582 | 6.83112 | 7.52403 | 8.922186 | 7.637571 | 8.12065 | 8.104887 | 7.258065 | 7.971495 |
| 3 | 6.623531 | 5.97723 | 6.281074 | 9.381917 | 9.392789 | 8.269804 | 9.330836 | 8.159393 | 8.021213 |
| x̅ | 5.908394 | 6.593928 | 6.67882 | 8.922186 | 8.364959 | 8.385814 | 8.871105 | 8.080329 | 8.203513 |
| SD | 0.520928 | 0.439925 | 0.597999 | 0.375369 | 0.747394 | 0.276323 | 0.545398 | 0.641539 | 0.29367 |
| Intra- | 8.816748 | 6.671668 | 8.953657 | 4.207139 | 8.934822 | 3.295125 | 6.14803 | 7.939516 | 3.579807 |
| batch | | | | | | | | | |
| CV % | | | | | | | | | |

| | | | |
|---|---|---|---|
| Inter- | 5.394649 | 3.01373 | 4.14312 |
| batch | | | |
| CV % | | | |

8.17 Comparative Test of Blocking ELISA Method and IFA

246 clinical samples were detected by the blocking ELISA method of antibody detection and method of antibodyvirus neutralization. After IFA test, there were 5 positive sera and 241 negative sera; using blocking ELISA, 6 positive sera and 240 negative sera were used. The overall concordance rate was 99.6%, a high concordance rate.

Further statistics of the two methods were perfolined to calculate the Kappa value. The results showed that the Kappa value was 0.91 (Kappa>0.75), indicating that the two detection methods were almost identical (Table 13). To sum up, the detection effect of the blocking ELISA method for detecting a SADS-CoV antibody established in the present disclosure was equivalent to that of the method for antibody detection by IFA.

TABLE 13
Blocking ELISA and IFA comparison results

| | | | | | | Concord- |
|---|---|---|---|---|---|---|
| Method | IFA positive | IFA negative | | Total | | ance rate |
| Blocking ELISA positive | 5 (a) | 1 | (b) | 6 | (a + b) | 99.6% |
| Blocking ELISA negative | 0 (c) | 240 | (d) | 240 | (c + d) | |
| Total | 5 (a + c) | 241 | (b + d) | 246 | (n) | |

| | |
|---|---|
| Kappa | 0.91 |

The calculation formula of the concordance rate and Kappa value is as follows:

coincidence rate=[(a+d)/n]*100;

P_{A}=(a+d)/n; Pₑ=[(a+b) (a+c) +(c+d) (b+d)]/n²; Kappa=(P_{A}−Pₑ)/(1−Pₑ).

The above embodiments are only preferred embodiments of the present disclosure and do not limit the scope of implementation of the present disclosure. Therefore, any equivalent changes or modifications made in accordance with the structures, features and principles described in the patent scope of the present disclosure should be included in the scope of the patent application of the present disclosure.
Sequence Listing Information:

DTD Version: V1_3

File Name: GWP20220400016_seqlist.xml

Software Name: WIPO Sequence

Software Version: 2.2.0

Production Date: 2022-11-14
General Information:

Current application/Applicant file reference: GWP20220400016

Earliest priority application/IP Office: CN

Earliest priority application/Application number: 202111449947.7

Earliest priority application/Filing date: 2021 Nov. 30

Applicant name: Institute of Urban Agriculture, Chinese Academy of Agricultural Sciences

Applicant name/Language: en

Invention title: BLOCKING ELISA KIT FOR DETECTING ANTIBODY TO SWINE ACUTE DIARRHEA SYNDROME CORONAVIRUS N PROTEIN (en)

Sequence Total Quantity: 11
Sequences:

Sequence Number (ID): 1

Length: 8

Molecule Type: AA

Features Location/Qualifiers:
source, 1..8
   mol_type, protein
   note, CDR1 in the heavy chain variable region
   organism, synthetic construct

Residues:

GYTFTDYA
8

Sequence Number (ID): 2

Length: 8

Molecule Type: AA

Features Location/Qualifiers:
source, 1..8
   mol_type, protein
   note, CDR2 in the heavy chain variable region
   organism, synthetic construct

Residues:

FSTYYGNA
8

Sequence Number (ID): 3

Length: 10

Molecule Type: AA

Features Location/Qualifiers:
source, 1 . . . 10
   mol_type, protein
   note, CDR1 in the light chain variable region
   organism, synthetic construct

Residues:

KSVSTSGYSY
10

Sequence Number (ID): 4

Length: 8

Molecule Type: AA

Features Location/Qualifiers:
source, 1..8
   mol_type, protein
   note, CDR3 in the light chain variable region
   organism, synthetic construct

Residues:

QHIRELTR
8

Sequence Number (ID): 5

Length: 120

Molecule Type: AA

Features Location/Qualifiers:
source, 1 . . . 120
   mol_type, protein
   note, heavy chain variable region of the mAb
   organism, synthetic construct

Residues:

QVQLKQSGAE LVRPGVSVKI SCKGSGYTFT DYAVHWVKQS
HAKSLEWIGV FSTYYGNANY 60
NQNFKGKATM TVDKSSNTAY MELARLTSED SAIYYCARGG
DYYGSSNVDY AMDYWGQGTS 120

Sequence Number (ID): 6

Length: 108

Molecule Type: AA

Features Location/Qualifiers:
source, 1 . . . 108
   mol_type, protein
   note, light chain variable region of the mAb
   organism, synthetic construct

Residues:

ENVLTQSPAS LAVSLGQRAT ISYRASKSVS TSGYSYMHWN
QQKPGQPPRL LIYLVSNLES 60
GVPARFSGSG SGTDFTLNIH PVEEEDAATY YCQHIRELTR
SEGGPSWS 108

Sequence Number (ID): 7

Length: 360

Molecule Type: DNA

Features Location/Qualifiers:
source, 1 . . . 360
   mol_type, other DNA
   note, DNA sequence encoding the heavy chain variable region of mAb
   organism, synthetic construct

Residues:

| | |
|---|---|
| caggtgcaac tgaagcagtc tggggctgag ctggtgaggc ctggggtctc agtgaagatt | 60 |
| tcctgcaagg gttctggcta cacattcact gattatgctg tgcactgggt gaagcagagt | 120 |
| catgcaaaga gtctagagtg gattggagtt tttagtactt actatggtaa tgctaactac | 180 |
| aaccagaact tcaagggcaa ggccacaatg accgtagaca aatcctccaa cacagcctat | 240 |
| atggaacttg ccagactgac atctgaggat tctgccatct attactgtgc aagaggaggg | 300 |
| gattactacg gtagtagcaa cgtagactat gctatggact actggggtca aggaacctca | 360 |

Sequence Number (ID): 8

Length: 327

Molecule Type: DNA

Features Location/Qualifiers:
source, 1 . . . 327
   mol_type, other DNA
   note, DNA sequence encoding the light chain variable region of mAb
   organism, synthetic construct

Residues:

| | |
|---|---|
| gaaaatgtgc tcacccagtc tcctgcttcc ttagctgtat ctctggggca gagggccacc | 60 |
| atctcataca gggccagcaa aagtgtcagt acatctggct atagttatat gcactggaac | 120 |
| caacagaaac caggacagcc acccagactc ctcatctatc ttgtatccaa cctagaatct | 180 |
| ggggtccctg ccaggttcag tggcagtggg tctgggacag acttcaccct caacatccat | 240 |
| cctgtggagg aggaggatgc tgcaacctat tactgtcagc acattaggga gcttacacgt | 300 |
| tcggaggggg gaccaagctg gagctga | 327 |

Sequence Number (ID): 9

Length: 375

Molecule Type: AA

Features Location/Qualifiers:
source, 1 . . . 375
   mol_type, protein
   note, SADS-CoV N protein
   organism, synthetic construct

Residues:

MATVNWGDAV EQAESRGRKR IPLSLFAPLR VIDGKNFWNV
MPRNGVPTGK GTPDQQIGYW 60
VEQKRWRMQK GQRKDQPSNW HFYYLGTGPH ADAPFRKRIQ
GVHWVAVDGA KTSPTGLGVR 120
NRNKEPATPQ FGFQLPPDLT VVEVTSRSAS RSQSRSRNQS
QSRSGAQTPR AQQPSQSVDI 180
VAAVKQALAD LGIASSQSRP QSGKNTPKPR SRAVSPAPAP
KPARKQMDKP EWKRVPNSEE 240
DVRKCFGPRS VSRNFGDSDL VQHGVEAKHF PTIAELLPTQ
AALAFGSEIT TKESGEFVEV 300
TYHYVMKVPK TDKNLPRFLE QVSAYSKPSQ IRRSQSQQDL
NADAPVFTPA PPATPVSQNP 360
AFLEEEVEMV DEIIN 375

Sequence Number (ID): 10

Length: 1128

Molecule Type: DNA

Features Location/Qualifiers:
source, 1 . . . 1128
   mol_type, other DNA
   note, DNA sequence encoding the SADS-CoV N protein
   organism, synthetic construct

Residues:

| | |
|---|---|
| atggccactg ttaattgggg tgacgctgtt gaacaggcgg aatctcgtgg tcgtaaaaga | 60 |
| attccattgt cactctttgc gcctttgcgt gttatagatg gcaaaaactt ttggaatgtc | 120 |
| atgcctagaa atggagttcc gacaggtaaa ggcactccag atcaacagat tggttattgg | 180 |
| gttgaacaaa aacgctggcg aatgcaaaaa ggccaacgta aagatcagcc ttctaactgg | 240 |
| cacttttatt accttggtac tggtcctcac gcagatgctc ctttcaggaa acggattcag | 300 |
| ggtgtgcatt gggtcgctgt tgacggtgct aaaactagcc ccacaggtct tggtgttcgc | 360 |
| aatcgtaaca aagaacctgc tacacctcag tttgggtttc aattaccacc agacctgact | 420 |
| gttgttgagg ttacttctag aagtgcttca cgttcacagt ctcgttctcg caatcaaagt | 480 |
| caaagccgca gtggtgctca gacacctcgt gctcaacagc cgtcacagtc tgttgacatt | 540 |
| gttgctgcag ttaaacaagc tttggcagac ttgggcatag cttctagcca gtccaggcct | 600 |
| caaagtggta aaaatacacc caaaccaaga agcagagctg tctcacctgc acctgcccct | 660 |
| aaaccggctc gtaagcagat ggacaaacct gaatggaagc gtgttcctaa ttctgaggag | 720 |
| gacgtgcgta aatgctttgg tcctcgctca gtttctagaa attttggtga cagtgacctc | 780 |
| gttcagcacg gtgttgaagc taagcacttt ccaacaattg ctgagttgct tccgacacaa | 840 |
| gctgcactag cctttggtag tgaaatcaca accaaagagt ctggtgaatt tgtagaagtc | 900 |
| acctatcact atgtaatgaa ggtccccaag actgataaaa atctacccag atttcttgag | 960 |
| caagtctcgg cttactctaa acccagtcaa attaggagat ctcaatctca acaagaccta | 1020 |
| aatgctgatg ccccagtgtt cactccggca cctccagcta ctccagtttc ccaaaatcct | 1080 |
| gcttttcttg aggaggaggt tgagatggtg gatgagatta ttaattag | 1128 |

Sequence Number (ID): 11

Length: 18

Molecule Type: AA

Features Location/Qualifiers:
source, 1 . . . 18
   mol_type, protein
   note, CDR3 in the heavy chain variable region
   organism, synthetic construct

Residues:

ARGGDYYG SSNVDYAMDY 18

END

What is claimed is:

1. A blocking ELISA kit for detecting an antibody to swine acute diarrhea syndrome coronavirus (SADS-CoV) N protein, comprising an ELISA plate and an enzyme-labeled antibody, wherein, the ELISA plate is coated with the SADS-CoV N protein, and the enzyme-labeled antibody is a mouse anti-SADS-CoV N protein monoclonal antibody (mAb) labeled with horseradish peroxidase (HRP);
a heavy chain variable region of the mouse anti-SADS-CoV N protein mAb comprises a CDR1 having the amino acid sequence set forth in SEQ ID NO: 1, a CDR2 having the amino acid sequence set forth in SEQ ID NO: 2, and a CDR3 having the amino acid sequence set forth in SEQ ID NO: 3;
a light chain variable region of the mouse anti-SADS-CoV N protein mAb comprises a CDR1 having the amino acid sequence set forth in SEQ ID NO: 4, a CDR2 having the amino acid sequence Leu-Val-Ser, and a CDR3 having the amino acid sequence set forth in SEQ ID NO: 5.

2. The blocking ELISA kit for detecting an antibody to SADS-CoV N protein according to claim 1, wherein the amino acid sequence of the SADS-CoV N protein is set forth in SEQ ID NO: 10.

3. The blocking ELISA kit for detecting an antibody to SADS-CoV N protein according to claim 1, wherein the amino acid sequence of the heavy chain variable region of the mouse anti-SADS-CoV N protein mAb is set forth in SEQ ID NO: 6; the amino acid sequence of the light chain variable region of the mouse anti-SADS-CoV N protein mAb is set forth in SEQ ID NO: 7.

4. The blocking ELISA kit for detecting an antibody to SADS-CoV N protein according to claim 3, wherein the DNA sequence encoding the heavy chain variable region of the mouse anti-SADS-CoV N protein mAb is set forth in SEQ ID NO: 8; the DNA sequence encoding the light chain variable region of the mouse anti-SADS-CoV N protein mAb is set forth in SEQ ID NO: 9.

5. The blocking ELISA kit for detecting an antibody to SADS-CoV N protein according to claim 1, wherein the kit further comprises a positive serum control and a negative serum control; the positive serum control is swine serum collected after artificial immunization with SADS-CoV; the negative serum control is swine serum without SADS-CoV pathogen.

6. The blocking ELISA kit for detecting an antibody to SADS-CoV N protein according to claim 1, wherein, the kit further comprises a coating solution, a blocking solution, a sample diluent, an enzyme-labeled antibody diluent, a washing solution, a color development solution and a stop solution.

7. A method for using the blocking ELISA kit for detecting an antibody to SADS-CoV N protein according to claim 1, comprising the following steps:
(1) coating: diluting a purified SADS-CoV N protein with a coating solution, subjecting an ELISA plate to coating overnight at 4° C., discarding liquid in the ELISA plate, and conducting washing and drying;
(2) blocking: adding a blocking solution, sealing the ELISA plate, discarding liquid in the ELISA plate, and conducting washing and drying;
(3) sample addition: adding a serum sample to be tested diluted with a sample diluent for reaction, setting negative control, positive control and blank control wells, discarding liquid in the ELISA plate, and conducting washing and drying;
(4) enzyme-labeled secondary antibody addition: adding an HRP-labeled mouse anti-SADS-CoV N protein mAb diluted with an enzyme-labeled antibody diluent for reaction, discarding a liquid in the plate, and conducting washing and drying;
(5) color development: adding TMB substrate to develop color in the dark;
(6) termination: adding 2M H₂SO₄ to stop the reaction;
(7) reading: measuring OD₄₅₀ by a microplate reader; and
(8) judgement: calculating the plaque inhibition (PI) based on a measured OD value according to PI=(OD value of negative control−OD value of serum to be tested)/OD value of negative control×100%;
a test sample with PI≥47.49613% is judged to be positive, a test sample with PI≤37.26795% is judged to be negative, and a test sample with 37.26795%<PI<47.49613% is judged to be suspected; a test sample judged to be suspected needs to be tested repeatedly; if PI is still lower than 47.49613% after a repeated test, it is judged to be serum antibody negative.

8. The method according to claim 7, wherein the SADS-CoV N protein is coated at 0.25 μg/mL.

9. The method according to claim 7, wherein the serum sample to be tested is diluted at a ratio of 1:4.

10. The method according to claim 7, wherein the HRP-labeled mouse anti-SADS-CoV N protein mAb is diluted at a ratio of 1:16,000.

11. The blocking ELISA kit for detecting an antibody to SADS-CoV N protein according to claim 5, wherein the amino acid sequence of the SADS-CoV N protein is set forth in SEQ ID NO: 10.

12. The blocking ELISA kit for detecting an antibody to SADS-CoV N protein according to claim 5, wherein the amino acid sequence of the heavy chain variable region of the mouse anti-SADS-CoV N protein mAb is set forth in SEQ ID NO: 6; the amino acid sequence of the light chain variable region of the mouse anti-SADS-CoV N protein mAb is set forth in SEQ ID NO: 7.

13. The blocking ELISA kit for detecting an antibody to SADS-CoV N protein according to claim 5, wherein the DNA sequence encoding the heavy chain variable region of the mouse anti-SADS-CoV N protein mAb is set forth in SEQ ID NO: 8; the DNA sequence encoding the light chain variable region of the mouse anti-SADS-CoV N protein mAb is set forth in SEQ ID NO: 9.

14. The blocking ELISA kit for detecting an antibody to SADS-CoV N protein according to claim 6, wherein the amino acid sequence of the SADS-CoV N protein is set forth in SEQ ID NO: 10.

15. The blocking ELISA kit for detecting an antibody to SADS-CoV N protein according to claim 6, wherein the amino acid sequence of the heavy chain variable region of the mouse anti-SADS-CoV N protein mAb is set forth in SEQ ID NO: 6; the amino acid sequence of the light chain variable region of the mouse anti-SADS-CoV N protein mAb is set forth in SEQ ID NO: 7.

16. The blocking ELISA kit for detecting an antibody to SADS-CoV N protein according to claim 6, wherein the DNA sequence encoding the heavy chain variable region of the mouse anti-SADS-CoV N protein mAb is set forth in SEQ ID NO: 8; the DNA sequence encoding the light chain variable region of the mouse anti-SADS-CoV N protein mAb is set forth in SEQ ID NO: 9.

17. The blocking ELISA kit for detecting an antibody to SADS-CoV N protein according to claim 6, wherein the kit further comprises a positive serum control and a negative serum control; the positive serum control is swine serum collected after artificial immunization with SADS-CoV; the negative serum control is swine serum without SADS-CoV pathogen.

18. The method for using the blocking ELISA kit for detecting an antibody to SADS-CoV N protein according to claim 7, wherein the amino acid sequence of the SADS-CoV N protein is set forth in SEQ ID NO: 10.

19. The method for using the blocking ELISA kit for detecting an antibody to SADS-CoV N protein according to claim 7, wherein the amino acid sequence of the heavy chain variable region of the mouse anti-SADS-CoV N protein mAb is set forth in SEQ ID NO: 6; the amino acid sequence of the light chain variable region of the mouse anti-SADS-CoV N protein mAb is set forth in SEQ ID NO: 7.

20. The method for using the blocking ELISA kit for detecting an antibody to SADS-CoV N protein according to claim 7, wherein the DNA sequence encoding the heavy chain variable region of the mouse anti-SADS-CoV N protein mAb is set forth in SEQ ID NO: 8; the DNA sequence encoding the light chain variable region of the mouse anti-SADS-CoV N protein mAb is set forth in SEQ ID NO: 9.
